(19) United States
(12) Patent Application Publication
(10) Pub. No.: US 2007/0112193 A1
(43) Pub. Date: May 17, 2007

(54) VALACYCLOVIR PROCESS

(76) Inventors: Mayur Devjibhai Khunt, Junagadh 362 260, Gujarat (IN); Naveen Kumar Reddy Keshava, Hyderabad 500 017, A.P. (IN); Ramachandra Reddy Bojja, Hyderabad 500 072, A.P. (IN); Prasada Raju Venkata Naga Kali Vara Veturi, Hyderabad 500 072, A.P. (IN); Prabhakara Sastry Venkata Ramakrishna Regella, Hyderabad 500 072, A.P. (IN); Somaiah Sripathi, Nalgonda, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/559,427

(22) Filed: Nov. 14, 2006

Related U.S. Application Data
(60) Provisional application No. 60/746,080, filed on May 1, 2006.
(60) Provisional application No. 60/824,947, filed on Sep. 8, 2006.

(30) Foreign Application Priority Data
Nov. 14, 2005 (IN) 1656/CHE/2005
Jul. 21, 2006 (IN) 1265/CHE/2006

(51) Int. Cl. C07D 473/10

(52) U.S. Cl. 544/276

(57) ABSTRACT

A process for preparing valacyclovir or a salt thereof.

### INTRODUCTION TO THE INVENTION

The present invention relates to a process for the preparation of valacyclovir and salts thereof. In an embodiment it relates to a process for the preparation of valacyclovir hydrochloride with reduced levels of impurities.

Valacyclovir hydrochloride has the chemical name L-valine, 2-[(2-amino-1,6-dihydro-6-oxo-9H-purin-9-yl)methoxy] ethyl ester, monohydrochloride and is depicted by structural Formula I.

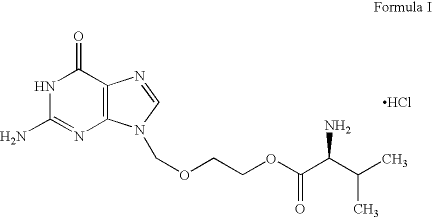

Valacyclovir is the L-valyl ester of acyclovir, an acyclic guanine nucleoside analogue, useful in the treatment of herpes simplex and varicella-zoster viral infections. It's hydrochloride salt is commercially available in pharmaceutical products sold using the trademark VALTREX as oral caplets equivalent to 500 mg and 1 g of valacyclovir.

U.S. Pat. No. 4,957,924 discloses valacyclovir, its salts, pharmaceutical compositions and a method of treatment using these compositions. It also describes a process for the preparation of valacyclovir hydrochloride by condensation of CBZ-Valine and acyclovir in dimethyl formamide with catalytic amount of 4-dimethyl amino pyridine (DMAP), to give protected valacyclovir. The CBZ group was removed by catalytic hydrogenation using Pd/C to give valacyclovir, which was subsequently converted into hydrochloride salt. The whole process can be depicted by the following reaction scheme A.

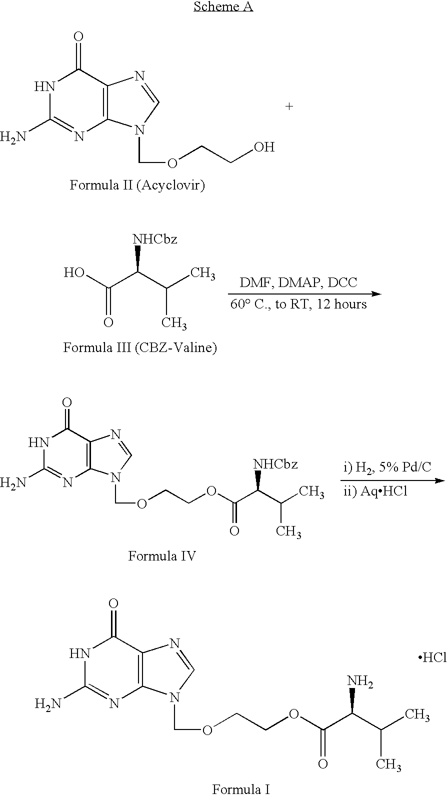

U.S. Pat. No. 6,849,737 discloses an alternate process for the preparation of valacyclovir hydrochloride by using an N-t-BOC protecting group instead of a CBZ group.

The aforementioned processes suffer from disadvantages during hydrochloride salt preparation due to formation of acyclovir as an impurity, leading to poor yields.

Hence, it is desirable to provide a simple, industrially feasible, inexpensive, and scaleable process for the synthesis of valacyclovir hydrochloride of Formula I.

### SUMMARY OF THE INVENTION

The present invention relates to a process for the preparation of valacyclovir and salts thereof.

In one aspect the present invention relates to a process for the preparation of valacyclovir or a salt thereof, comprising:

i) reacting 2-amino-1,9-dihydro-9-[(2-hydroxyethoxy)methyl]-6H-purin-6-one of Formula II with carbobenzyloxy-L-valine of Formula III to form 2-[(2-amino-1,6-dihydro-6-oxo-9h-purin-9yl)methoxy] ethyl n-[(benzyloxy)carbonyl]-L-valinate of Formula IV;

ii) reducing 2-[(2-amino-1,6-dihydro-6-oxo-9h-purin-9yl)methoxy] ethyl n-[(benzyloxy)carbonyl] I-valinate of Formula IV with hydrogen in the presence of palladium on aluminum oxide to form valacyclovir; and

iii) converting valacyclovir into a pharmaceutically acceptable salt.

In another aspect the present invention relates to a process for the preparation of valacyclovir hydrochloride, comprising:

a) providing a solution of valacyclovir in dimethyl formamide;

b) reacting with hydrochloric acid;

c) adding an antisolvent and recovering the valacyclovir hydrochloride.

In yet another aspect the present invention provides a process for reducing the metal content in valacyclovir or its salts, comprising:

a) providing a solution of valacyclovir or its salt in a suitable solvent;

b) treating the solution of step a) with a resin; and

c) adding an antisolvent and recovering the valacyclovir or its salt having a reduced metal content.

An embodiment of the invention includes a process for preparing valacyclovir or a salt thereof, comprising reacting 2-amino-1,9-dihydro-9-[(2-hydroxyethoxy) methyl]-6H-purin-6-one with carbobenzyloxy-L-valine to form an intermediate, and reducing an intermediate with hydrogen in the presence of a palladium on aluminum oxide catalyst to form valacyclovir.

Another embodiment of the invention includes a process for preparing valacyclovir hydrochloride, comprising combining a solution of valacyclovir in dimethylformamide with hydrochloric acid and adding an antisolvent for valacyclovir hydrochloride.

A further embodiment of the invention includes a A process for preparing valacyclovir hydrochloride dihydrate, comprising adding acetone to an aqueous solution of valacyclovir hydrochloride.

A still further embodiment of the invention includes a purification process comprising contacting a solution comprising valacyclovir or a salt thereof with a resin, and recovering a solution comprising purified valacyclovir or a salt thereof.

### DETAILED DESCRIPTION OF THE INVENTION

The present invention relates to a process for preparation of valacyclovir and salts thereof.

In one aspect the present invention relates to a process for the preparation of valacyclovir or a salt thereof, comprising:

i) reacting 2-amino-1,9-dihydro-9-[(2-hydroxyethoxy)methyl]-6H-purin-6-one of Formula II with carbobenzyloxy-L-valine of Formula III 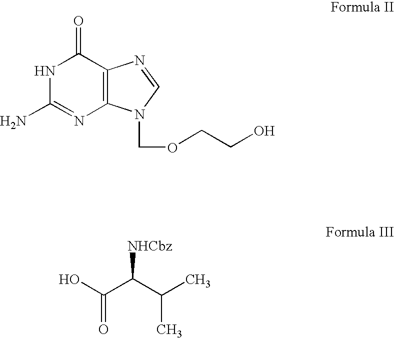
where “bz” is a benzyl group, to form 2-[(2-amino-1,6-dihydro-6-oxo-9h-purin-9yl)methoxy]ethyl N-[(benzyloxy)carbonyl] L-valinate of Formula IV;

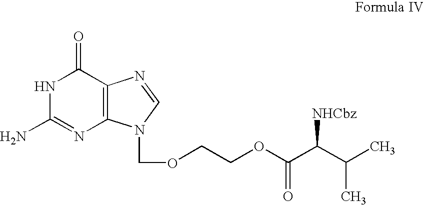

ii) reducing 2-[(2-amino-1,6-dihydro-6-oxo-9h-purin-9yl)methoxy] ethyl N-[(benzyloxy)carbonyl] L-valinate of Formula IV with hydrogen in the presence of palladium on aluminum oxide to form valacyclovir; and

iii) optionally, converting the valacyclovir into a pharmaceutically acceptable salt.

Step i) involves a reaction of 2-amino-1,9-dihydro-9-[(2-hydroxyethoxy)methyl]-6H-purin-6-one of Formula II with carbobenzyloxy-L-valine of Formula III to form 2-[(2-amino-1,6-dihydro-6-oxo-9h-purin-9yl)methoxy] ethyl n-[(benzyloxy)carbonyl]-L-valinate of Formula IV, in the presence of a suitable reagent.

Suitable solvents used in the process of step i) include but are not limited to: esters such as ethyl acetate, n-propylacetate, isopropyl acetate, n-butyl acetate, tertiary butyl acetate and the like; halogenated solvents such as dichloromethane, ethylene dichloride, chloroform, carbon tetrachloride and the like; ether solvents such diethyl ether, tertiary butyl ether, methyl tertiary butyl ether, 1,4-dioxane and the like; nitrile solvents such as acetonitrile, propionitrile and the like; hydrocarbon solvents such as toluene, xylene, n-hexane, n-heptane, cyclohexane, and the like; aprotic polar solvents such as N,N-dimethyl formamide (DMF), dimethylsulfoxide (DMSO), N,N-dimethyl acetamide (DMA) and the like; and mixtures thereof or their combinations with water in various proportions without limit.

Suitable reagents which can be used for the condensation include but are not limited to carbodiimide compounds such as 1-ethyl-3-(3-dimethylaminopropyl) carbodiimide (EDCI), dicyclohexyl carbodiimide (DCC), and diisopropyl carbodiimide (DIPCDI).

Suitable temperatures for conducting the reaction range from about −10° C. to about 30° C., or from about 0° C. to 20° C.

Step ii) involves reducing 2-[(2-amino-1,6-dihydro-6-oxo-9h-purin-9yl)methoxy] ethyl N-[(benzyloxy)carbonyl] L-valinate of Formula IV with hydrogen in the presence of palladium on aluminum oxide, to form valacyclovir.

Palladium on aluminum oxide used in the present invention for hydrogenation increases the rate of reaction, consistently yielding valacyclovir with reduced levels of impurities. It is also suitable for commercialization as it can be recycled easily.

The reaction can be carried out in presence of solvents and those can include, but are not limited to, either non polar solvents or polar solvents such as: water; alcoholic solvents such as methanol, ethanol, propanol, butanol and the like; esters such as ethyl acetate, n-propylacetate, isopropyl acetate, n-butyl acetate, tertiary butyl acetate and the like; halogenated solvents such as dichloromethane, ethylene dichloride, chloroform, carbon tetrachloride and the like; ether solvents such diethyl ether, tertiary butyl ether, methyl tertiary butyl ether, 1,4-dioxane and the like; nitrile solvents such as acetonitrile, propionitrile and the like; hydrocarbon solvents such as toluene, xylene, n-hexane, n-heptane, cyclohexane, and the like; aprotic polar solvents such as N,N-dimethylformamide (DMF), dimethylsulfoxide (DMSO), N,N-dimethyl acetamide (DMA) and the like; and mixtures thereof or their combinations with water in various proportions without limit.

The amount of palladium coated on the aluminum oxide can range from about 5% to about 10% w/w.

Suitable temperatures for conducting the reaction range from about −10° C. to about 150° C., or from about 0° C. to 100° C. The duration of maintenance of the reaction mixture at the reaction temperatures for reaction completion will vary considerably depending on the solvent and temperature chosen, for example about 1 to 48 hours, or about 1 to 36 hours, or about 5 to 24 hours, or longer.

Step iii) involves reaction of valacyclovir obtained from step ii) with a suitable pharmaceutically acceptable acid to form a pharmaceutically acceptable acid addition salt.

Suitable acids that can be used include but are not limited to: inorganic acids such as hydrochloric acid, hydrobromic acid, sulphuric acid, and the like; and organic acids such as oxalic acid, trifluoroacetic acid, tartaric acid, formic acid, acetic acid, para-toluene sulfonic acid and the like.

In another aspect the present invention relates to a process for the preparation of valacyclovir hydrochloride comprising:

a) providing a solution of valacyclovir in dimethyl formamide;

b) reacting with hydrochloric acid;

c) adding an antisolvent and recovering the valacyclovir hydrochloride.

The step of providing a solution of valacyclovir in dimethyl formamide includes dissolving valacyclovir in dimethyl formamide or the solution may be obtained directly from a reaction in which valacyclovir is formed.

When the solution is prepared by dissolving valacyclovir in dimethyl formamide, any form of valacyclovir such as any crystalline or amorphous form, including any salts, solvates and hydrates may be utilized for preparing the solution.

Suitable temperatures for providing a solution may range from about 10 to 60° C., or about 20 to 40° C., or room temperature.

The valacyclovir solution concentration in step a) may range from about 1% to 50% by w/v, or about 5% to 30%, or about 15% to 25% by w/v.

Step b) involves reacting the solution of step a) with hydrochloric acid to form valacyclovir hydrochloride.

The hydrochloride that can be used includes but is not limited to methanolic hydrochloride, isopropanolic hydrochloride, ethyl acetate hydrochloride, aqueous hydrochloric acid, gaseous hydrogen chloride and the like.

An amount of hydrochloric acid that is required for converting valacyclovir to valacyclovir hydrochloride may be determined by measuring the reaction mixture pH, as hydrochloric acid is added. Suitably, an ending reaction mixture pH may range from about 3 to about 6, or about 4 to about 6, or about 4 to 5.

The reaction mixture pH affects formation of impurities. If the reaction mixture pH is below 4, then formation of impurities is enhanced.

Suitably, hydrochloric acid may be added at temperatures ranging from about −10° C. to about 60° C., or from about 0° C. to 30° C.

Step c) involves adding an antisolvent and recovering the valacyclovir hydrochloride.

Suitable antisolvents that can be used in step c) include any solvent or mixture of solvents in which valacyclovir hydrochloride has a limited solubility. Examples include: ketones such as acetone, methyl ethyl ketone, ethyl isopropyl ketone, methyl propyl ketone, and the like; nitriles such as acetonitrile, propionitrile, and the like; esters such as ethyl acetate, propyl acetate, and the like; alcohols such as methanol, ethanol, propanol, butanol, and the like; ethers such as tetrahydrofuran, diethyl ether, diisopropyl ether, methyl tert-butyl ether, and the like; and mixtures thereof in any proportions.

Suitably antisolvent is added at temperatures ranging from about −10 ° C. to about 30° C., or from about 0° C. to 15° C., to precipitate the solid product.

The valacyclovir hydrochloride solid can be recovered by using conventional solid-liquid separation techniques, such as decantation, centrifugation, gravity filtration, vacuum filtration, and the like.

The isolated solid product can optionally be dried using any technique, such as fluid bed drying (FBD), aerial drying, oven drying or other techniques known in the art at temperatures of about 25-100° C., or 50-60° C., with or without application of vacuum and/or under inert conditions.

In yet another aspect the present invention provides a process for reducing metal content in valacyclovir or its salt, comprising:

a) providing a solution of valacyclovir or its salt in a suitable solvent;

b) treating the solution of step a) with a resin;

c) adding antisolvent and recovering the valacyclovir or its salt with a reduced metal content.

The step of providing a solution of valacyclovir or its salt in a suitable solvent includes dissolving valacyclovir or its salt in suitable solvent or such a solution may be obtained directly from a reaction in which valacyclovir or its salt is formed.

Suitable solvents that can be used for providing a solution of valacyclovir or its salt include but are not limited to polar solvents such as: water; aprotic polar solvents such as N,N-dimethyl formamide (DMF), dimethylsulfoxide (DMSO), N,N-dimethyl acetamide (DMA) and the like; and mixtures thereof in various proportions without limit.

When the solution is prepared by dissolving valacyclovir or its salt in suitable solvent, any form of valacyclovir or its salt such as any crystalline or amorphous form, including any salts, solvates and hydrates may be utilized for preparing the solution.

Suitable temperatures for providing solution may range from about 10 to 60° C., or about 20 to 40° C., or room temperature.

The valacyclovir or its hydrochloride salt solution concentration in step a) may range from about 1% to 50%, or about 5% to 30%, or about 15% to 25%, by w/v.

Step b) involves treating the solution of step a) with a resin to adsorb the metal impurities.

Selection of the resin depends upon the properties of the resin (pore size, grain size, surface area, polarity of the surface and solubility index), type of material to be purified, and the level and nature of impurities present.

Resins which can be used for the purpose of the present invention include, T-5201 R, T-42 MP, T-40 MP, T-54, t-310, CXO-10, T-66 MP, T-38, Thermax CH-97 and Thermax CH-96, all manufactured by Thermax Limited, India; AMBERLITE® IR-120NA, AMBERLITE IRC-76, AMBERLITE IRC-86, AMBERLITE IR-200C, resins manufactured by Rohm and Haas Corporation, Philadelphia, Pa. U.S.A.; INDION® 204, INDION 214, INDION 234, INDION 254, and INDION 414, manufactured by Ion Exchange India Limited; and the like.

Particular examples of strong acid cation exchange resins that are useful in the present invention generally have —COOH or —SO₃⁻functional groups, including crosslinked polyacrylics, and polystyrene resins having varying degrees of crosslinking with divinybenzene or benzenesulfonic acid functionality, the latter type being shown below structurally in its sodium salt form:

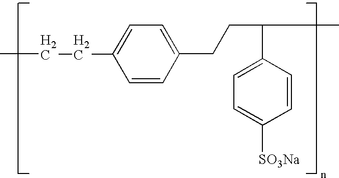

Resin that is used for metals removal may be activated by washing with water before it is used in the process of the present invention. After treatment by mixing resin with a solution, resin is separated from the solution by techniques such as gravity filtration, vacuum filtration and the like. Alternatively, the solution can be passed through a bed, such as a column, of resin particles; this technique can be conducted in a manner that avoids any further solid-liquid separation requirement.

Quantities of resin that are used in the present invention can range from about 2% to 15%, or about 10%, of the weight of valacyclovir or its salt in the solution to be treated.

Step c) involves adding an antisolvent and recovering the valacyclovir or its hydrochloride salt with a reduced metal content.

Suitable antisolvents that can be used in step c) include any solvent or mixture of solvents in which valacyclovir hydrochloride has a limited solubility. Examples include: ketones such as acetone, methyl ethyl ketone, ethyl isopropyl ketone, methyl propyl ketone, and the like; nitriles such as acetonitrile, propionitrile, and the like; esters such as ethyl acetate, propyl acetate, and the like; alcohols such as methanol, ethanol, propanol, butanol, and the like; ethers such as tetrahydrofuran, diethyl ether, diisopropyl ether, methyl tert-butyl ether, and the like; and mixtures thereof in any proportions.

Suitably, an antisolvent is added at temperatures ranging from about −10° C. to about 30° C., or from about 0° C. to 15° C., to precipitate the solid product.

The valacyclovir or its hydrochloride salt solid can be recovered using conventional solid-liquid separation techniques, such as decantation, centrifugation, gravity filtration, pressure filtration, vacuum filtration, and the like.

The isolated solid product can optionally be dried using any technique, such as fluid bed drying, aerial drying, oven drying or other techniques known in the art at temperatures of about 25 to 100° C., or 50 to 60 ° C., with or without application of vacuum and/or under inert conditions.

Valacyclovir or its salt prepared according to the process of the present invention has a palladium content less than about 10 ppm, or about less than 5 ppm, and an aluminum content less than about 20 ppm, or about less than 10 ppm, as measured by atomic absorption spectroscopy (“AAS”).

Valacyclovir hydrochloride prepared according to the process of the invention is characterized by its XRPD pattern having prominent peaks approximately at about 3.5, 9.4, 10.5, 10.8, and 13.2, ±0.2 degrees 2θ, using Cu Kα-1 radiation.

Valacyclovir hydrochloride prepared according to the process of the present invention contains about 8 to 11%, or about 9 to 10%, of water by weight when measured using the Karl Fischer method.

Valacyclovir hydrochloride prepared according to the process of the present invention contains about less than or equal to about 0.1% by weight of impurity A (guanine impurity); less than or equal to about 1% by weight of impurity B (acyclovir impurity), less than or equal to about 0.1% by weight of impurity E; less than or equal to about 0.1% by weight of impurity H (alanine impurity); less than or equal to about 0.1% by weight of impurity I (O-acetyl impurity); less than or equal to about 0.1% by weight of impurity J (isolucine impurity);less than or equal to about 0.1% by weight of impurity M (N-formyl impurity) and less than or equal to about 5% of total impurities including Formula IV, Formula III and other enantiomers, as measured by high performance liquid chromatography (“HPLC”).

Valacyclovir hydrochloride obtained by the process of the invention is analyzed using the HPLC method described in European Pharmacopeia Vol 18. No. 2, April 2006, at pages 309 and 314.

Additionally two new impurities have been identified, which are formed if the pH is adjusted below 4 during the preparation of valacyclovir hydrochloride.

Valacyclovir hydrochloride prepared according to the process of the present invention contains about less than or equal to about 0.1% by weight of an N-acetyl valacyclovir impurity and less than or equal to about 0.1% by weight of the 1.96 RRT impurity as measured by HPLC.

As used herein “N-acetyl valacyclovir impurity” refers to 2-acetyl amino-3-methyl-butyric acid 2-(2-amino-6-oxo-1,6-dihydro-purin-9-ylmethoxy)-ethyl ester of Formula VI.

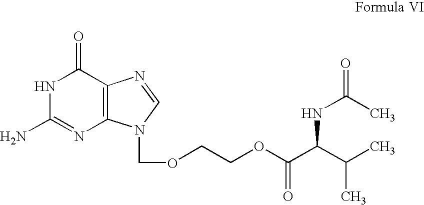

One unidentified impurity observed by HPLC is referred to herein by its relative retention time (“RRT”) with respect to valacyclovir or its salt. It consistently appears in the above-referenced HPLC procedure at an RRT of about 1.96 (and is herein referred to as the “1.96 RRT impurity”).

The term “RRT” as used herein is intended to indicate the relative retention time of the particular impurity against a pure valacyclovir or salt standard (assigned an RRT value of 1) during an HPLC analysis, an examples of a useful HPLC analytical method being described herein below. A relative standard deviation that is equal to or less than 5% for a population of 6 injections is acceptable while referring to RRT values.

The above-mentioned two process-related impurities are analyzed by the high performance liquid chromatography (HPLC) method using an Inertsil ODS 3V, 250×4.6 mm×5 μm column with the parameters in Table 1:

TABLE 1

| Flow rate | 1.0 ml/minute | | | |
|---|---|---|---|---|
| Detector | 254 nm | | | |
| Injection load | 20 μl | | | |
| Column Temperature | 40° C. | | | |
| Gradient conditions | | | Mobile | Mobile |
| | | | phase A | phase A |
| | Interval | | (percent | (percent |
| | (min) | Flow | v/v) | v/v) |
| | 0.00 | 1.0 | 100 | 0 |
| | 2.5 | 1.0 | 100 | 0 |
| | 15.00 | 1.0 | 85 | 15 |
| | 30.0 | 1.0 | 55 | 45 |
| | 40.0 | 1.0 | 10 | 90 |
| | 55.0 | 1.0 | 10 | 90 |
| | 15.0 | 1.0 | 85 | 15 |
| | 60.0 | 1.0 | 100 | 0 |
| | 65.0 | 1.0 | 100 | 0 |

| | |
|---|---|
| Mobile phase | Buffer Preparation: 3.4 g of potassium |
| | dihydrogen phosphate dissolved 1000 ml |
| | of water. Adjust the pH to 6.6 with triethylamine. |
| | Mobile phase A: Mix 900 ml of buffer |
| | and 100 ml of methanol, filter and de- |
| | gas through a filter having 0.45 μm |
| | pores. |
| | Mobile phase B: Mix well 500 ml of |
| | buffer and 200 ml of acetonitrile and |
| | 300 ml of methanol, filter and de-gas |
| | through a filter having 0.45 μm pores. |

Valacyclovir hydrochloride obtained by the process of the present invention contains less than about 5000 ppm, or less than about 3000 ppm, or less than about 1000 ppm, of total residual organic solvents and less than about 200 ppm, or less than about 100 ppm, or less than about 50 ppm, of individual residual organic solvents as determined by gas chromatography.

Valacyclovir hydrochloride obtained by the process of the present invention has a particle size distribution with 25% of particles having sizes greater than 250 μm and 90% of the particles having sizes less than 420 μm.

Valacyclovir hydrochloride obtained by the process of the present invention has a bulk density of about 0.4 g/ml to about 0.8 g/ml. The bulk densities are determined using Test 616 “Bulk Density and Tapped Density,” United States Pharmacopeia 24, pages 1913-4 (United States Pharmacopeial Convention, Inc., Rockville, Md., 1999).

Valacyclovir hydrochloride obtained by the process of the present invention has a Hausner ratio in the range of about 1.1 to 1.5.

The Hausner ratio is a measure of inter-particle friction and the potential powder arch or bridge strength and stability (Hausner, H. H. Friction conditions in a mass of metal powders. International Journal of Powder Metallurgy 1967, 3(4), pp. 7-13). It has been widely used to estimate the flow properties of powders, blends, granules and other such particles or aggregates and is expressed as the ratio of tapped bulk density to the untapped bulk density of the substance.

In another aspect of the present invention, there is provided Valacyclovir hydrochloride having high purity, which is well suited for use in pharmaceutical compositions, as well as a related method of treatment.

These and other specific aspects and embodiments of this invention are described in further detail by the examples below, which examples are not intended to limit the scope of the appended claims in any manner.

### EXAMPLE 1

Preparation of 2-[(2-AMINO-1,6-DIHYDRO-6-OXO-9H-PURIN-9YL)METHOXY] ETHYL N-[(BENZYLOXY)CARBONYL] L-VALINATE (Formula IV)

50 g of N-[(benzyloxy)carbonyl] L-valine (CBZ-L-valine) of Formula III and 500 ml of dimethyl formamide (DMF) were charged into a round bottom flask followed by stirring with simultaneous cooling to 15° C. over a period of 10 minutes. To the obtained clear solution 68.6 g of dicyclohexyl carbodiimide dissolved in 100 ml of dimethyl formamide was slowly added over a period of 45 minutes followed by stirring for 15 minutes at 15° C. 50 g of 9-((2-hyroxyethoxy)methyl)-2-amino-1H-purin-6(9H)-one (acyclovir) of Formula II and 4.066 g of dimethylaminopyridine were charged into the reaction mass and subjected to stirring for about 6 hours at 15 ° C. After completion of the reaction, the reaction mass was filtered and the solids washed with 100 ml of dimethyl formamide. The resultant filtrate was subjected to distillation under vacuum at 80° C. until 80% of the volume was distilled. The resultant filtrate was transferred into another flask and 300 ml of water was added at 30° C. followed by heating to 70° C. for a period of 30 minutes. The mixture was cooled to 20° C. and the separated solid was filtered, followed by washing the solid with 100 ml of water. The solid material was subjected to suction drying to afford the 256 g of wet solid title compound.

256 g of the above-obtained wet material was taken into a round bottom flask along with 750 ml of methanol and 225 ml of water, followed by heating to 80° C. with simultaneous stirring for 4 hours. The obtained mass was cooled to a temperature of 35 ° C. with simultaneous stirring. The solid obtained was filtered and washed with a mixture of 338 ml methanol and 178 ml water. Finally the obtained solid material was subjected to suction drying followed by drying under vacuum at 55° C. to afford 88.7 g (87.2%) of the title compound.

Purity: 99.3 weight % by HPLC.

### EXAMPLE 2

Prepration of Valacyclovir Hydrochloride

100 g of 2-[(2-amino-1,6-dihydro-6-oxo-9h-purin-9yl)methoxy] ethyl n-[(benzyloxy)carbonyl] I-valinate of Formula IV was added into a stainless steel vessel containing 1 liter of DMF. 10 g of 5% palladium on aluminum oxide was added to the above reaction mixture and the resultant reaction mixture was maintained at about 30 ° C. while applying 4 kg/cm² hydrogen pressure. After completion, the reaction mixture was subjected to distillation at 80° C. by applying a vacuum of about 400 mm Hg to remove about 680 ml of DMF. The resultant concentrated solution was cooled to about 10° C. Reaction mixture pH was adjusted to about 3.8 using of 18 ml of 36% aqueous hydrochloric acid with stirring for a period of 15 minutes followed by addition of 250 ml of water. The resultant solution was filtered through a flux calcined diatomaceous earth (“Hyflow”) bed followed by washing with 50 ml of water. The resultant filtrate was transferred into another round bottom flask followed by the addition of 4.5 liters of acetone and subjected to stirring for a period of 1 hour. The solid obtained was filtered and washed with 50 ml of acetone. 95 g of the wet solid thus obtained was used for further purification.
1^{st} Purification: 89.5 g of the above-obtained wet solid was charged into a round bottom flask containing 447 ml of dimethyl formamide and stirred for a period of 9 hours at 30° C. 224 ml of isopropyl alcohol was added to the solution and subjected to stirring for a period of 15 minutes. The separated solid was filtered and washed with 45 ml of isopropyl alcohol followed by subjecting to suction drying over a period of 60 minutes to afford 128 g of wet compound, which was further subjected to a second purification.
2^{nd} purification: 82.5 g of the above-obtained wet compound and 82.5 ml of water were added into a round bottom flask and heated to 55° C. with simultaneous stirring for 15 minutes. The mass was cooled to 0° C. followed by addition of 619 ml acetone with simultaneous stirring for a period of 60 minutes. The separated solid was filtered and subjected to suction drying for 10 minutes to afford 76 g of wet compound, which was further subjected to a third purification.

3^{rd} purification: The above obtained 76 g of wet compound and 360 ml of dimethyl formamide were taken into a dry round bottom flask and stirred for about 8 hours. 180 ml of isopropyl alcohol was added to the formed solution and stirred for about 1 hour. The separated solid was filtered and washed with 36 ml of isopropyl alcohol and subjected to suction drying for 10 minutes. The obtained solid was dried under vacuum at about 60° C. for about 10 hours to afford 38 g of title compound.

TABLE 2
HPLC purity of Valacyclovir Hydrochloride (values in weight percent, all
samples dried before analysis)

| | Before | After 1^{st} | After 2^{nd} | After 3^{rd} |
|---|---|---|---|---|
| Compound | Purification | Purification | Purification | Purification |
| Valacyclovir | 96.60 | 98.36 | 99.32 | 99.70 |
| hydrochloride | | | | |
| Impurity A | 0.009 | 0.004 | 0.004 | ND |
| Impurity B | 1.076 | 0.496 | 0.402 | 0.141 |
| Impurity H | ND | ND | ND | ND |
| Impurity I | 0.135 | 0.061 | 0.047 | 0.015 |
| Impurity M | 0.209 | 0.099 | 0.074 | 0.024 |
| Impurity J | ND | 0.007 | 0.007 | 0.005 |
| Maximum | 0.065 | 0.041 | 0.040 | 0.036 |
| single unknown | | | | |
| impurity | | | | |
| Total | 1.68 | 0.85 | 0.68 | 0.30 |
| impurities | | | | |

ND = Not detected

### EXAMPLE 3

Preparation of Valacyclovir Hydrochloride

30 g of 2-[(2-amino-1,6-dihydro-6-oxo-9h-purin-9yl)methoxy] ethyl n-[(benzyloxy)carbonyl]-L-valinate of Formula IV and 3 g of 5% palladium on aluminum oxide were charged into a stainless steel vessel containing 300 ml of DMF. The reaction mixture was maintained at 30° C. by applying 4.2 kg/cm² of hydrogen pressure with stirring for a period of 4 hours. After reaction completion, catalyst was removed by filtering through Hyflow. The filtrate was distilled at 80° C. to remove 200 ml of the solvent. The resultant concentrated solution was subjected to cooling to 5° C. and 3 ml of 36% aqueous hydrochloric acid was slowly added with stirring to adjust the pH to 4. 160 ml of isopropyl alcohol was added to the above with simultaneous stirring at 5° C. over a period of 1 hour. The obtained slurry was filtered and the solid washed with 16 ml of isopropyl alcohol followed by subjecting to suction drying. The wet material was transferred into a round bottom flask containing 225 ml of DMF and subjected to stirring for a period of 13 hours. The obtained solid product was filtered, washed with 15 ml of isopropyl alcohol and subjected to suction drying over a period of 30 minutes. Finally the obtained wet solid was dried at 60° C. over a period of 7 hours to afford 16 g of valacyclovir hydrochloride.

### EXAMPLE 4

Preparation of Valacyclovir Hydrochloride

50 g of 2-[(2-amino-1,6-dihydro-6-oxo-9h-purin-9yl)methoxy] ethyl n-[(benzyloxy)carbonyl] I-valinate of Formula IV was taken into an autoclave containing 500 ml of DMF and subjected to stirring. 5 g of palladium on aluminum oxide was added to the above mixture and the resultant mixture was maintained at about 30° C. with the application of 4.0 kg/cm² hydrogen pressure. After the completion of the reaction the reaction mixture was subjected to distillation at 70° C. by applying a vacuum of 400 mm Hg to remove about 350 ml of the solvent. The resultant concentrated solution was cooled to 10° C. pH was adjusted to 3.8 using of 9.3 ml of 36% aqueous hydrochloric acid with stirring for a period of 15 minutes. The mixture was cooled to 5° C. followed by addition of 125 ml of water. The resultant clear solution was filtered through Hyflow followed by washing with 50 ml of water. The filtrate was transferred to another flask.

1.4 g of TULSION T-63 resin (procured from Thermax Ltd.) was taken into a beaker followed by the addition of 30 ml of water and stirring for 10 minutes. Water was decanted from the mixture and the above process repeated two more times. The resin was filtered and washed with 30 ml of water. The resin was added to 21 ml of the filtrate obtained above and subjected to stirring for 1 hour. The suspension was filtered and the obtained filtrate was passed through a 0.45 μm filter paper. The filtrate was transferred into another round bottom flask followed by the addition of 11.25 ml of acetone and subjected to stirring for a period of 1 hour. The solid obtained was filtered and washed with 12.5 ml of acetone under vacuum. The wet compound thus obtained was subjected to drying in an oven at a temperature of 70° C. for about 3 hours to afford 6.5 g of the valacyclovir hydrochloride.

TABLE 3
Metal content by AAS

| Metal | Content |
|---|---|
| Palladium | 5.41 ppm |
| Aluminum | 6.55 ppm |

### EXAMPLE 5

Preparation of Valacyclovir Hydrochloride

15 kg of 2-[(2-amino-1,6-dihydro-6-oxo-9h-purin-9yl)methoxy] ethyl n-[(benzyloxy)carbonyl] I-valinate of Formula IV was dissolved in 135 liters of DMF and taken into a reactor. 0.75 kg of 5% palladium on alumina in 15 liters of DMF was added to the above solution at 30° C. The reaction mixture was maintained at 33° C. and 4.2 kg/cm² of hydrogen pressure applied for a period of about 4.5 hours. After reaction completion, the mixture was subjected to distillation at 77° C. by applying a vacuum of 680 mm Hg to remove about 120 liters of solvent. The resultant concentrate was cooled to 4° C. for a period of 1.5 hours and pH was adjusted to 3.75 using 2 liters of 36% aqueous hydrochloric acid at 2.9° C. over a period of 5 hours. 37.5 liters of water was added and the temperature was slowly raised to 20° C. for a period of 30 minutes. The mixture was filtered through a Hyflow bed followed by washing the bed with 7.5 liters of water. The filtrate was transferred into another reactor and 31.5 liters of acetone was added at a temperature of 9° C. over a period of 1 hour and stirred for about 1 hour. The suspension was filtered followed by washing the solid with 7.5 liters of acetone, and the solid was subjected to suction drying for a period of 11 hours to afford 16.2 kg of wet compound which was further subjected to purification.
1^{st} purification: The wet compound was transferred into a reactor containing 75 liters of DMF and subjected to stirring for a period of 7 hours, 15 minutes at 30° C. 37.5 liters of isopropyl alcohol was added to the above reaction solution at 29° C. over a period of 55 minutes. The suspension was filtered and the solid washed with 5 liters of isopropyl alcohol followed by suction drying for a period of 1 hour, 45 minutes. The wet material was transferred into a drier and subjected to drying at 60° C. by applying a vaccum of 600 mm Hg for a period of 5 hours, 15 minutes followed by cooling to 32° C. to afford 7.4 kg of the title compound.
2^{nd} purification: 5.0 kg of valacyclovir hydrochloride obtained from the above purification was placed into a reactor containing 50 liters of DMF and subjected to stirring for a period of 8 hours at 31° C. 25 liters of isopropyl alcohol was added to the above solution and maintained for a period of 1 hour at 29° C., and the reaction mass was filtered followed by washing the solid with 5 liters of isopropyl alcohol. The wet compound was subjected to spin drying for a period of 2 hours, 20 minutes. Finally the obtained solid material was subjected to drying at a temperature of 60° C. over a period of 12.5 hours by applying a vacuum of 600 mm Hg, followed by cooling to 29° C. to afford 4.5 kg of the title compound.

Impurity A: Not Detected.

Impurity B : 0.172 weight %.

Impurity H: Not Detected

Impurity I: 0.027 weight %

Impurity M: 0.044 weight %.

Impurity J : Not Detected.

Maximum single unknown Impurity: 0.031%

Total Impurities: 0.336 weight %.

### EXAMPLE 6

Preparation of Valacyclovir Hydrochloride Dihydrate

6 liters of water was taken into reactor followed by the addition of 3 kg of valacyclovir hydrochloride obtained from Example 4 with simultaneous stirring and heating to 51 ° C., followed by stirring for a period of 10 minutes until dissolution was obtained. The mass was filtered through a Hyflow bed and washed with 3 liters of hot water. The filtrated was transferred into another reactor and subjected to cooling to 4° C. for a period of 1 hour, 22 minutes and then 60 liters of acetone were added followed by maintaining for a period of 1 hour. The mass was filtered and the solid washed with 3 liters of acetone and subjected to spin drying for a period of 3 hours. The obtained solid was dried under vacuum at about 60° C. for 11 hours to afford 3 kg of the title compound.

Moisture Content: 9.32% w/w by the Karl Fischer method

Impurity A: Not Detected.

Impurity B: 0.33 weight %.

Impurity H: Not Detected

Impurity I: 0.024 weight %

Impurity M: 0.013 weight %.

Impurity J: Not Detected.

Maximum single unknown Impurity: 0.036%

Total Impurities: 0.41 weight %.

Residual Solvents by gas chromatography:
Isopropanol, methanol, toluene, DMF and acetic acid: Not detected
Acetone: 3 ppm
Ethyl acetate: 5 ppm

### EXAMPLE 7

Preparation of Valacyclovir Hydrochloride Using Acetonitrile as Antisolvent

15 g of valacyclovir was charged into a clean, dry round bottom flask containing 30 ml of DMF at 30° C. Cooled the contents to 2° C. and adjusted the pH to 2 using 5.5 ml of 36% aqueous hydrochloric acid. The contents were stirred for 10 minutes. 150 ml of acetonitrile was added into the mass at about 0-5° C. and stirred for 20 minutes. Filtered the solid and washed with 30 ml of acetonitrile. Dried the solid at 50° C. under vacuum of 100 torr for about 6 hours to get 17.5 g of the title compound.

### EXAMPLE 8

Preparation of Valacyclovir Hydrochloride Using Acetone as Antisolvent

1.0 g of valacyclovir was charged into a clean, dry round bottom flask containing 2 ml of DMF at room temperature. Cooled the contents to 50° C. and stirred for 5 minutes. The pH was adjusted to 2 using 0.4 ml of 36% aqueous hydrochloric acid and stirred for 10 minutes at 5° C. 15 ml of acetone was charged into the reaction mass at 50° C. and stirred for 20 minutes. Filtered the solid and washed with 2 ml of acetone. Dried the solid at 50° C. under a vacuum of 100 torr for 6 hours to get 1 g of the title compound.

### EXAMPLE 9

Preparation of Valacyclovir Hydrochloride Using Ethyl Acetate as Antisolvent

1.0 g of valacyclovir was charged into a clean, dry round bottom flask containing 2 ml of DMF at room temperature. Cooled the contents to 5° C. and stirred for 10 minutes. The pH was adjusted to 2 using 0.4 ml of 36% aqueous hydrochloric acid and the mixture stirred for 10 minutes. 15 ml of ethyl acetate was charged into the mass at about 50° C. and stirred for 20 minutes. Filtered the solid and washed with 2 ml of ethyl acetate. Dried the solid at 50° C. under a vacuum of 100 torr for 6 hours to get 1 g of the title compound.

### EXAMPLE 10

Preparation of Valacyclovir Hydrochloride Using THF as Antisolvent

1.0 g of valacyclovir was charged into a clean, dry round bottom flask containing 2 ml of DMF at room temperature. Cooled the contents to 15° C. and stirred for 15 minutes. The pH was adjusted to 2 using 0.4 ml of 36% aqueous hydrochloric acid and the mixture stirred for 10 minutes. 10 ml of tetrahydrofuran (THF) was charged into the reaction mass at about 15° C. and stirred for 20 minutes. Filtered the solid and washed with 2 ml of THF. Dried the solid at 50 ° C. under a vacuum of 100 torr for 6 hours to get 1 g of the title compound.

### EXAMPLE 11

Preparation of Valacyclovir Hydrochloride Using IPA as Antisolvent

1.0 g of valacyclovir was charged into a clean, dry round bottom flask containing 2 ml of DMF at room temperature. Cooled the contents to 3° C. and stirred for 10 minutes. The pH was adjusted to 2 using 0.4 ml of 36% aqueous hydrochloric acid and stirred for 10 minutes. 30 ml of isopropanol (IPA) was charged into the reaction mass at about 5° C. and stirred for 20 minutes. Filtered the solid and washed with 2.4 ml of IPA. Dried the solid at 60° C. under a vacuum of 100 torr for 6 hours to get 0.9 g of the title compound.

### EXAMPLE 12

Purification of Valacyclovir Hydrochloride Using Resin

1 g of TULSION T-63 resin (procured from Thermax Ltd.) and 10 ml of water were added into a beaker followed by stirring for 10 minutes. Water was decanted from the suspension and the process repeated two more times. The obtained suspension was filtered and washed with 5 ml of water. 10 g of valacyclovir hydrochloride and 20 ml of water were taken into another round bottom flask and heated to a temperature of 65 ° C., with simultaneous stirring. To the solution the resin suspension was added and the mixture stirred for 20 minutes. The suspension was filtered through a 0.45 μm filter paper and washed with 10 ml of water. The filtrate was transferred into another round bottom flask and 200 ml of acetone added under stirring. The obtained suspension was stirred for about 1 hour. The solid was filtered and washed with 5 ml of acetone. Wet solid thus obtained was dried at a temperature of 60° C. to afford 9.7 g of the valacyclovir hydrochloride.

TABLE 4
Metal content by AAS

| Metal | Content |
|---|---|
| Palladium | 1.48 ppm |
| Aluminum | 4.24 ppm |

### EXAMPLE 13

Preparation of N-Acetyl Valacyclovir

10 g of valacyclovir and 100 ml of acetic acid were added into a clean round bottom flask and subjected to cooling at 10° C. To the reaction mixture 6 ml of acetic anhydride was slowly added over a period of 10 minutes. The resultant mixture was stirred at 17° C. for 22 hours. After completion of the reaction, the solvent was expelled completely by purging with nitrogen gas. 100 ml of water was charged to the mass and stirred for 30 minutes at 18° C. Separated solid was filtered and the solid was washed with 10 ml of water. The solid obtained was dried at 30° C. to afford 4.2 g of the title compound.

### EXAMPLE 14

Isolation of 1.96 RRT Impurity

35 g of valacyclovir and 20 ml of DMF were charged into a clean and dry round bottom flask followed by stirring for about 15 minutes at 5° C. The mixture pH was adjusted to 2 by addition of 3.66 ml of 36% aqueous hydrochloric acid over about 15 minutes followed by addition of 100 ml of acetonitrile. The resultant mixture was stirred at about 5° C. for 30 minutes. The separated solid was filtered and the solid was washed with 20 ml of acetonitrile. The solid obtained was dried at about 50° C. under vacuum to afford 12 g of valacyclovir hydrochloride. The filtrate obtained above was concentrated to form a residue.

The above-obtained residue was subjected to preparative high performance liquid chromatography [Inertsil PREP-C8, 250×20 mm ID column manufactured by GL Sciences] using petroleum ether and ethyl acetate in the ratio of 8.6:1.4 as the eluent and the fractions of title compound were collected. The fractions were combined and the compound was isolated by conventional techniques to afford 2.5 g of pure title compound.

1. A process for preparing valacyclovir or a salt thereof, comprising reacting 2-amino-1,9-dihydro-9-[(2-hydroxyethoxy)methyl]-6H-purin-6-one with carbobenzyloxy-L-valine to form an intermediate, and reducing an intermediate with hydrogen in the presence of a palladium on aluminum oxide catalyst to form valacyclovir.

2. The process of claim 1, wherein reacting to form an intermediate occurs in the presence of a carbodiimide compound.

3. The process of claim 1, wherein reacting to form an intermediate occurs in the presence of dicyclohexyl carbodiimide.

4. The process of claim 1, wherein a catalyst contains about 5 to about 10 weight percent palladium.

5. The process of claim 1, wherein valacyclovir is isolated and then reacted with hydrochloric acid to form a hydrochloride salt.

6. A process for preparing valacyclovir hydrochloride, comprising combining a solution of valacyclovir in dimethylformamide with hydrochloric acid and adding an antisolvent for valacyclovir hydrochloride.

7. The process of claim 6, wherein hydrochloric acid is added to a solution of valacyclovir, until a pH about 4 to about 6 is obtained.

8. The process of claim 6, wherein hydrochloric acid is added to a solution of valacyclovir, until a pH about 4 to about 5 is obtained.

9. The process of claim 6, wherein an antisolvent comprises at least one ketone, nitrile, ester, alcohol, ether, or a mixture thereof.

10. The process of claim 6, wherein an antisolvent comprises isopropanol, tetrahydrofuran, acetonitrile, acetone, ethyl acetate, or a mixture of any two or more thereof.

11. A process for preparing valacyclovir hydrochloride dihydrate, comprising adding acetone to an aqueous solution of valacyclovir hydrochloride.

12. A purification process comprising contacting a solution comprising valacyclovir or a salt thereof with a resin, and recovering a solution comprising purified valacyclovir or a salt thereof.

13. The process of claim 12, wherein a quantity of resin is about 2 to about 15 percent of a weight of valacyclovir or a salt thereof.

14. The process of claim 12, wherein a resin is a cation exchange resin.

15. The process of claim 12, wherein a resin is a strong acid cation exchange resin.

16. The process of claim 12, further comprising combining a solution of valacyclovir in dimethylformamide with hydrochloric acid and adding an antisolvent for formed valacyclovir hydrochloride.

17. The process of claim 16, wherein hydrochloric acid is added to a solution of valacyclovir, until a pH about 4 to about 6 is obtained.

18. The process of claim 16, wherein hydrochloric acid is added to a solution of valacyclovir, until a pH about 4 to about 5 is obtained.

19. The process of claim 16, wherein an antisolvent comprises at least one ketone, nitrile, ester, alcohol, ether, or a mixture thereof.

20. The process of claim 16, wherein an antisolvent comprises isopropanol, tetrahydrofuran, acetonitrile, acetone, ethyl acetate, or a mixture of any two or more thereof.
